(19) United States
(12) Patent Application Publication
(10) Pub. No.: US 2015/0258085 A1
(43) Pub. Date: Sep. 17, 2015

(54) Treating Tumors of the Central Nervous System

(71) Applicant: THE REGENTS OF THE UNIVERSITY OF CALIFORNIA, Oakland, CA (US)

(72) Inventor: Krystof S. Bankiewicz, Oakland, CA (US)

(21) Appl. No.: 14/435,719

(22) PCT Filed: Oct. 18, 2013
(86) PCT No.: PCT/US13/65734

§ 371 Date: Apr. 14, 2015

Related U.S. Application Data
(60) Provisional application No. 61/716,027, filed on Oct. 19, 2012.

(51) Int. Cl. A61K 31/4745; A61K 9/127; A61K 31/4188

(52) U.S. Cl. CPC A61K 31/4745; A61K 31/4188; A61K 9/127

(57) ABSTRACT

A synergistic therapeutic effect is obtained in CNS cancer patients treated concomitantly with a first antineoplastic agent and a second antineoplastic agent, wherein one or both antineoplastic agents are administered by convection enhanced delivery. Combinations of interest include, without limitation, CED delivery of a topoisomerase inhibitor, e.g., topotecan, and systemic delivery of a triazene, e.g. temozolomide.

### GOVERNMENT RIGHTS

This invention was made with Government support under grant CA118816 awarded by the National Institutes of Health. The Government has certain rights in this invention.

### FIELD OF THE INVENTION

The present invention relates to treatment of tumors of the central nervous system comprising the concomitant delivery of at least two antineoplastic agents, wherein one of the antineoplastic agents is administered by convection enhanced delivery.

### BACKGROUND OF THE INVENTION

Of all brain tumors diagnosed each year in the United States, about half are malignant gliomas and result in death within 18 months. Gliomas originate from glial cells, most often astrocytes, and may occur anywhere in the brain or spinal cord, including the cerebellum, brain stem, or optic chiasm. Gliomas can be divided into two groups based on their growth characteristics: low-grade gliomas and high-grade gliomas. Low-grade gliomas are usually localized and grow slowly over a long period of time. Examples of low-grade gliomas include astrocytomas, oligodendrogliomas, pilocytic astrocytomas. Over time, most of these low-grade gliomas dedifferentiate into more malignant high-grade gliomas that grow rapidly and can easily spread through the brain. Examples of high-grade gliomas include anaplastic astrocytoma and glioblastoma multiforme.

Despite advances in conventional therapies for malignant gliomas which include surgical removal, radiation therapy, and chemotherapy as well as combinations thereof, malignant gliomas continue to be associated with a poor prognosis. For example, systemic delivery of therapeutics is usually associated with systemic side effects while achieving only marginal therapeutic concentrations in the central nervous system (CNS), and thus the efficacy of systemic treatment is limited. In a 2009 phase II clinical trial studying the therapeutic effect of systemic concomitant delivery of the topoisomerase I inhibitor, irinotecan, and the alkylating agent, temozolomide (TMZ), in subjects with newly diagnosed glioblastoma, the clinical outcome with the combination therapy was comparable to TMZ alone, while the combination appeared more toxic and poorly tolerated. Quinn et al. (2009) J. Neurooncol. 95(3):393-400, entitled “Phase II trial of temozolomide (TMZ) plus irinotecan (CPT-11) in adults with newly diagnosed glioblastoma multiforme before radiotherapy.”

Thus, there remains a need for more effective therapeutics with an acceptable safety profile to treat the growth and metastasis of a variety of cancers of the CNS, including gliomas.

### SUMMARY OF THE INVENTION

Disclosed herein are methods that combine therapeutic agents for a surprising synergistic effect in the treatment of central nervous system cancer. The methods of the invention provide for a period of concomitant delivery of two antineoplastic agents, when one or both of the antineoplastic agents are administered by convection enhanced delivery (CED).

Aspects of the invention include methods for inhibiting the growth of a CNS tumor, reducing a CNS tumor, killing CNS tumor cells, and/or treating a patient having a CNS tumor. The methods comprise administering to the patient therapeutically effective amounts of a first antineoplastic agent and a second antineoplastic agent, wherein at least the first antineoplastic agent is administered by CED and wherein the concomitant administration of the first and second antineoplastic agents inhibits the growth of a CNS tumor, reduces a CNS tumor, kills CNS tumor cells and/or treats a patient having a CNS tumor.

In one embodiment, the first antineoplastic agent is a topoisomerase I inhibitor, which includes topoisomerase I/II inhibitors, and is preferably a camptothecan or a derivative thereof. In a preferred embodiment, the topoisomerase inhibitor is liposomally encapsulated. Camptothecin derivatives of interest include those selected from the group consisting of 9-aminocamptothecin, 7-ethylcamptothecin, 10-hydroxycamptothecin, 9-nitrocamptothecin, 10,11 methlyenedioxycamptothecin, 9-amino-10,11-methylenedioxycamptothecin 9-chloro-10,11-methylenedioxycamptothecin, irinotecan, topotecan, 7-(4-methylpiperazinomethylene)-10,11-ethylenedioxy-20(S)-camptothecin, 7-(4-methylpiperazinomethylene)-10,11-methylenedioxy-20(S)-camptothecin and 7-(2-(N-isopropylamino)ethyl)-(20S)-camptothecin. In another embodiment, the camptothecin derivative is selected from the group consisting of irinotecan, topotecan, (7-(4-methylpiperazinomethylene)-10,11-ethylenedioxy-20(S)-camptothecin, 7-(4-methylpiperazinomethylene)-10,11-methylenedioxy-20(S)-camptothecin or 7-(2-(N-isopropylamino)ethyl)-(20S)-camptothecin. In a particularly preferred embodiment, the camptothecin derivative is topotecan encapsulated in a liposomal formulation.

In one embodiment, the second antineoplastic agent is an alkylating agent. Preferably, the second antineoplastic agent is a triazene selected from the group consisting of dacarbazine and TMZ. In a particularly preferred embodiment, the second antineoplastic agent is TMZ.

In particular, the methods of the invention provide a synergistic therapeutic effect when temozolomide (TMZ) is administered concomitantly with a liposomally encapsulated topoisomerase inhibitor administered by CED. In some embodiments the topoisomerase inhibitor is topotecan. In some embodiments TMZ is administered systemically, including without limitation oral delivery. In other embodiments TMZ is administered by CED.

In specific embodiments of the invention, TMZ is administered in accordance with a conventional protocol, wherein the protocol further comprises at least one concomitant administration of a liposomally encapsulated topoisomerase I inhibitor by CED, i.e., during at least a portion of the period of time in which TMZ is administered, the liposomally encapsulated topoisomerase I inhibitor is administered by CED. The concomitant administration of the topoisomerase I inhibitor may be during any phase of the TMZ treatment protocol, e.g. during all or part of the initial phase of treatment, e.g. the initial week, 2 week, 3 week, 4 week, 5 week, 6 week, etc. phase; during all or part of the maintenance phase, e.g. following the initial phase and an optional pause in treatment and during any or all of the cycles of maintenance treatment; or both the initial phase and the maintenance phase. The TMZ may be administered systemically or by CED. Additional therapeutic regimens are not excluded, e.g. a concomitant initiation phase may also comprise radiation, other chemotherapeutic agents; and the like.

In some embodiments the CNS cancer is a glioma, including glioblastoma multiforme (GBM), anaplastic astrocytoma, e.g. relapsed anaplastic astrocytoma; oligodendroglioma; and the like.

### BRIEF DESCRIPTION OF THE DRAWINGS

FIG. 1. Administration of liposomally encapsulated topotecan (TopoCED™) via CED, (20 μl, 1 mg/ml) delivered via CED into the brain together with systemic administration of TMZ (50 mg/kg/day) leads to a marked increase in survival in a rat tumor model compared to treatment with TopoCED™ alone or with systemic TMZ alone. The survival curve shows a synergistic increase when the TopoCED™ and TMZ are administered concomitantly.

FIG. 2. Comparison of equivalent doses (20 μl, 1 mg/ml) of TopoCED™ and free topotecan, when administered via CED in combination with systemic TMZ (50 mg/kg/day). Rats were xenografted with a human glioblastoma line, and treated with TMZ on days 0 and 4, and with the respective topotecan formulation at days 0, 3, 10 and 13. Animals were sacrificed at day 22. It can be seen that the free topotecan is much more toxic than TopoCED™.

FIG. 3. TopoCED™ in a canine astrocytoma grade III. The treatment resulted in close to 80% coverage of the tumor in this canine patient demonstrating potential for local delivery of liposomal topotecan.

FIG. 4. Upregulation of Topoisomerase I following treatment with TMZ.

### DETAILED DESCRIPTION OF THE EMBODIMENTS

The methods of the invention provide a synergistic therapeutic effect when an alkylating agent, e.g. TMZ, is administered for a period of time concomitantly with a liposomally encapsulated topoisomerase inhibitor, e.g. TopoCED™, administered by CED. While the invention is not limited by the underlying basis for the synergistic effect, it is believed the therapeutic enhancement is due in part to the upregulation of topoisomerase I by the alkylating agent, (see Mainwaring et al., “Sequential temozolomide followed by topotecan in the treatment of glioblastoma multiforme.” Proc Am Soc Clin Oncol. 2001; 20:abstr 245). Concomitant administration of a topoisomerase I inhibitor therefore increases the efficacy of the alkylating agent, while providing a second, intrinsic therapeutic agent. However, topoisomerase inhibitors, e.g. topotecan, do not cross the blood brain barrier at tolerable systemic drug levels, and they may cause local toxicity when administered to the CNS in native form Therefore, it is only with the CED delivery of liposomally encapsulated drug that adequate doses can be delivered locally in brain and brain tumors to realize the potential for synergy.

### Tumors of the Central Nervous System

As used herein, a “CNS tumor” or “tumor of the CNS” refers to a primary or malignant tumor of the CNS of a subject, e.g., the aberrant growth of cells within the CNS. The aberrantly growing cells of the CNS may be native to the CNS or derived from other tissues.

Gliomas are the most common primary tumors of the CNS. Glioblastoma multiforme (GBM) is the most frequent and the most malignant type of glioma. There is a much higher incidence of GBM in adults than in children. According to the Central Brain Tumor Registry of the United States statistical report, GBM accounts for about 20% of all brain tumors in the USA (CBTRUS, 1998-2002). Other tumors of the CNS include, but are not limited to, other gliomas, including astrocytoma, including fibrillary (diffuse) astrocytoma, pilocytic astrocytoma, pleomorphic xanthoastrocytoma, and brain stem glioma, oligodendroglioma, ependymoma and related paraventricular mass lesions, neuronal tumors, poorly differentiated neoplasms, including medulloblastoma, other parenchymal tumors, including primary brain lymphoma, germ cell tumors, pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma).

### Antineoplastic Agents

Suitable antineoplastic agents to be used in the present invention include, but are not limited to, natural antineoplastic agents, alkylating agents, antimetabolites, angiogenesis inhibitors, differentiating reagents, small molecule enzymatic inhibitors, biological response modifiers, and anti-metastatic agents.

Natural antineoplastic agents comprise antimitotic agents, antibiotic antineoplastic agents, camptothecin analogues, and enzymes. Antimitotic agents suitable for use herein include, but are not limited to, vinca alkaloids like vinblastine, vincristine, vindesine, vinorelbine, and their analogues and derivatives. They are derived from the Madagascar periwinkle plant and are usually cell cycle-specific for the M phase, binding to tubulin in the microtubules of cancer cells. Other antimitotic agents suitable for use herein are the podophyllotoxins, which include, but are not limited to etoposide, teniposide, and their analogues and derivatives. These reagents predominantly target the G2 and late S phase of the cell cycle.

Also included among the natural antineoplastic agents are the antibiotic antineoplastic agents. Antibiotic antineoplastic agents are antimicrobial drugs that have anti-tumor properties usually through interacting with cancer cell DNA. Antibiotic antineoplastic agents suitable for use herein include, but are not limited to, belomycin, dactinomycin, doxorubicin, idarubicin, epirubicin, mitomycin, mitoxantrone, pentostatin, plicamycin, and their analogues and derivatives.

The natural antineoplastic agent classification also includes camptothecin analogues and derivatives which are suitable for use herein and include camptothecin, topotecan, and irinotecan. These agents act primarily by targeting the nuclear enzyme topoisomerase I. Another subclass under the natural antineoplastic agents is the enzyme, L-asparaginase and its variants. L-asparaginase acts by depriving some cancer cells of L-asparagine by catalyzing the hydrolysis of circulating asparagine to aspartic acid and ammonia.

### Alkylating Agents

Alkylating agents are known to act through the alkylation of macromolecules such as the DNA of cancer cells, and are usually strong electrophiles. This activity can disrupt DNA synthesis and cell division. Examples of alkylating reagents suitable for use herein include nitrogen mustards and their analogues and derivatives including, cyclophosphamide, ifosfamide, chlorambucil, estramustine, mechlorethamine hydrochloride, melphalan, and uracil mustard. Other examples of alkylating agents include alkyl sulfonates (e.g. busulfan), nitrosoureas (e.g. carmustine, lomustine, and streptozocin), triazenes (e.g. dacarbazine and TMZ), ethylenimines/methylmelamines (e.g. altretamine and thiotepa), and methylhydrazine derivatives (e.g. procarbazine). Included in the alkylating agent group are the alkylating-like platinum-containing drugs comprising carboplatin, cisplatin, and oxaliplatin.

### Topoisomerase Inhibitors

DNA topoisomerases are enzymes essential for the relaxation of DNA during a number of critical processes, including replication, transcription, and repair. There are two types of topoisomerases; topoisomerase I and topoisomerase II. Camptothecin and related compounds are the most important inhibitors of topoisomerase I, including irinotecan and topotecan. In addition, several topoisomerase I inhibitors that are structurally related to camptothecin are in development, including BNP1350, SN38, 9-amino-camptothecin, lurtotecan, gimatecan, several homocamptothecins, such as diflomotecan, and several conjugates, usually via the 20S hydroxy or a 10 hydroxy, with, for example, carboxymethyldextran, poly-L-gutamic acid, polyethylene glycol and the like, such as T-0128, DX-310, CT-2106 and Protecan.

The term “topoisomerase II inhibitors” as used herein includes, but is not limited to the antracyclines doxorubicin (including liposomal formulation), epirubicin, idarubicin and nemorubicin, the anthraquinones itoxantrone and losoxantrone, and the podophillotoxi\acute{η}es etoposide and teniposide.

### Antimetabolites

Antimetabolic antineoplastic agents structurally resemble natural metabolites, and are involved in normal metabolic processes of cancer cells such as the synthesis of nucleic acids and proteins. They differ enough from the natural metabolites so that they interfere with the metabolic processes of cancer cells. Suitable antimetabolic antineoplastic agents to be used in the present invention can be classified according to the metabolic process they affect, and can include, but are not limited to, analogues and derivatives of folic acid, pyrimidines, purines, and cytidine. Members of the folic acid group of agents suitable for use herein include, but are not limited to, methotrexate (amethopterin), pemetrexed and their analogues and derivatives. Pyrimidine agents suitable for use herein include, but are not limited to, cytarabine, floxuridine, fluorouracil (5-fluorouracil), capecitabine, gemcitabine, and their analogues and derivatives. Purine agents suitable for use herein include, but are not limited to, mercaptopurine (6-mercaptopurine), pentostatin, thioguanine, cladribine, and their analogues and derivatives. Cytidine agents suitable for use herein include, but are not limited to, cytarabine (cytosine arabinodside), azacitidine (5-azacytidine) and their analogues and derivatives.

### Angiogenesis Inhibitors

Angiogenesis inhibitors work by inhibiting the vascularization of tumors. Angiogenesis inhibitors encompass a wide variety of agents including small molecule agents, antibody agents, and agents that target RNA function. Examples of angiogenesis inhibitors suitable for use herein include, but are not limited to, ranibizumab, bevacizumab, SU11248, PTK787, ZK222584, CEP-7055, angiozyme, dalteparin, thalidomide, suramin, CC-5013, combretastatin A4 Phosphate, LY317615, soy isoflavones, AE-941, interferon alpha, PTK787/ZK 222584, ZD6474, EMD 121974, ZD6474, BAY 543-9006, celecoxib, halofuginone hydrobromide, bevacizumab, their analogues, variants, or derivatives.

### Differentiating Agents

Differentiating agents inhibit tumor growth through mechanisms that induce cancer cells to differentiate. One such subclass of these agents suitable for use herein includes, but is not limited to, vitamin A analogues or retinoids, and peroxisome proliferator-activated receptor agonists (PPARs). Retinoids suitable for use herein include, but are not limited to, vitamin A, vitamin A aldehyde (retinal), retinoic acid, fenretinide, 9-cis-retinoid acid, 13-cis-retinoid acid, all-trans-retinoic acid, isotretinoin, tretinoin, retinyl palmitate, their analogues and derivatives. Agonists of PPARs suitable for use herein include, but are not limited to, troglitazone, ciglitazone, tesaglitazar, their analogues and derivatives.

### Small Molecule Enzymatic Inhibitors

Certain small molecule therapeutic agents are able to target the tyrosine kinase enzymatic activity or downstream signal transduction signals of certain cell receptors such as epidermal growth factor receptor (“EGFR”) or vascular endothelial growth factor receptor (“VEGFR”). Such targeting by small molecule therapeutics can result in anti-cancer effects. Examples of such agents suitable for use herein include, but are not limited to, imatinib, gefitinib, erlotinib, lapatinib, canertinib, ZD6474, sorafenib (BAY 43-9006), ERB-569, and their analogues and derivatives.

### Biological Response Modifiers

Certain protein or small molecule agents can be used in anti-cancer therapy through either direct anti-tumor effects or through indirect effects. Examples of direct-acting agents suitable for use herein include, but are not limited to, differentiating reagents such as retinoids and retinoid derivatives. Indirect-acting agents suitable for use herein include, but are not limited to, agents that modify or enhance the immune or other systems such as interferons, interleukins, hematopoietic growth factors (e.g. erythropoietin), and antibodies (monoclonal and polyclonal).

### Anti-Metastatic Agents

The process whereby cancer cells spread from the site of the original tumor to other locations around the body is termed cancer metastasis. Certain agents have anti-metastatic properties, designed to inhibit the spread of cancer cells. Examples of such agents suitable for use herein include, but are not limited to, marimastat, bevacizumab, trastuzumab, rituximab, erlotinib, MMI-166, GRN163L, hunter-killer peptides, tissue inhibitors of metalloproteinases (TIMPs), their analogues, derivatives and variants.

### Delivery and Dosage Forms

In the methods disclosed herein, at least the first antineoplastic agent, e.g. a topoisomerase inhibitor, is administered in a liposome encapsulated form by CED. The second antineoplastic agent may be administered by CED or systemically, e.g. as an oral dosage form. Accordingly, another aspect of the present invention relates to formulations and routes of administration for the pharmaceutical composition(s) comprising a first antineoplastic agent and a second antineoplastic agent. Such pharmaceutical compositions can be used to treat a cancer of the CNS.

Pharmaceutical compositions can be used in the preparation of individual, single unit dosage forms. Pharmaceutical compositions and dosage forms of the invention comprise a first antineoplastic and/or a second antineoplastic agent, or a pharmaceutically acceptable salt, solvate, hydrate, stereoisomer, clathrate, or prodrug thereof. Pharmaceutical compositions and dosage forms of the invention can further comprise one or more excipients.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient. In some embodiments, the first antineoplastic agent is in a liposomal formulation and the second antineoplastic agent is in an oral dosage form.

### Liposomal Formulations

As used herein, “liposome” refers to a lipid bilayer membrane containing an entrapped aqueous volume. Liposomes may be unilamellar vesicles having a single membrane bilayer or multilamellar vesicles having multiple membrane bilayers separated from each other by an aqueous layer. Generally, the liposomal bilayer is composed of two lipid monolayers having a hydrophobic “tail” region and a hydrophilic “head” region. The structure of the membrane bilayer is such that the hydrophobic (non-polar) “tails” of the lipid monolayers orient toward the center of the bilayer while the hydrophilic (polar) “heads” orient toward either the entrapped aqueous volume or the extraliposomal aqueous environment.

“Liposomal formulations” are understood to be those in which part or all of the therapeutic drug and/or diagnostic agent is encapsulated inside the liposomes. “Consisting essentially of” as used herein in reference to liposomal formulations refers to liposomes having the recited lipid components only, and no additional lipid components.

“Phospholipid” is understood to mean an amphiphile derivative of glycerol in which one of its hydroxyl groups is esterified with phosphoric acid and the other two hydroxyls are esterified with long-chain fatty acids, which may be equal or different from each other.

A saturated phospholipid will be that whose fatty acids only have simple (not multiple) covalent carbon-carbon bonds.

A neutral phospholipid will generally be one in which another phosphoric acid hydroxyl is esterified by an alcohol substituted by a polar group (usually hydroxyl or amine) and whose net charge is zero at physiological pH.

An anionic phospholipid will generally be one in which another phosphoric acid hydroxyl is esterified by an alcohol substituted by a polar group and whose net charge is negative at physiological pH.

The meaning of the expression “charged saturated phospholipid”, as well as including charged saturated phospholipids, also includes other amphiphile compounds whose net charge is different from zero. Such amphiphile compounds include, but are not limited to, long chain hydrocarbonate derivatives, substituted by a polar group (for example amine) and derivatives of fatty acids. Liposomal formulations described herein, e.g., pharmaceutical compositions comprising such formulations, may be formed in a variety of ways, including by active or passive loading methodologies. For example, one or more therapeutic drug(s) and/or diagnostic agent(s) may be encapsulated using a transmembrane pH gradient loading technique. General methods for loading liposomes with therapeutic drugs through the use of a transmembrane potential across the bilayers of the liposomes are well known to those in the art (e.g., U.S. Pat. Nos. 5,171,578; 5,077,056; and 5,192,549).

In some embodiments, a pegylated liposome is used for encapsulation of the drug. In other embodiments, a non-pegylated liposome is used for encapsulation.

The lipid components used in forming the non-PEGylated liposomes may be selected from a variety of vesicle forming lipids, typically including phospholipids and sterols (e.g., U.S. Pat. Nos. 5,059,421 and 5,100,662). For example, phospholipids derived from egg yolk, soybean or other vegetable or animal tissue, such as phosphatidylcholines, phosphatidylethanolamines, phosphatidic acid, phosphatidylserines, phosphatidylinositols, phosphatidylglycerols, sphingomyelins, etc.; mixtures thereof such as egg yolk phospholipid, soybean phospholipid, etc.; hydrogenation products thereof; and synthetic phospholipids such as dipalmitoylphosphatidlcholines, distearoylphosphatidylcholines, distearoylphosphatidylglycerols or the like may be used.

In one embodiment, non-PEGylated anionic liposomes are employed comprising a mixture of two or more non-PEGylated lipids, e.g., a neutral phospholipid and an anionic phospholipid. In one embodiment, the neutral phospholipid is chosen from the group composed of derivatives of phosphatidylcholine and their combinations, for example dipalmitoylphosphatidylcholine (DPPC), distearoylphosphatidylcholine (DSPC), dimyristoylphosphatidylcholine (DMPC) and their combinations. In one embodiment, the anionic phospholipid is selected from a group composed of derivatives of phosphatidylglycerol, dipalmitoyl phosphatidyl glycerol (DPPG), phosphatidylserine, phosphatidylinositol, phosphatidic acid and their combinations, for example, distearoyl phosphatidyl glycerol (DSPG) and a mixture of phosphatidylserine esters with different saturated fatty acids (PS). For stabilization of liposomes and other purposes, a sterol (e.g., cholesterol), α-tocopherol, dicetyl phosphate, stearylamine or the like may also be added.

A pegylated phospholipid may comprise PEG covalently bound with the hydrophilic moiety (polar head) of a phospholipid. The end of the PEG chain that has not been bound with the phospholipid may also be a hydroxyl group or an ether with a short chain such as with methyl or ethyl or an ester with a short chain such as with acetic acid or lactic acid. PEG chain length in the PEG-bound phospholipid molecule is desirably in the range of 5-1000 moles, more preferably 40-200 moles in terms of the average degree of polymerization. In order to produce a covalent bond between PEG and a phospholipid a reaction-active functional group is necessary at the polar moiety of the phospholipid. The functional group includes amino group of phosphatidylethanolamine, hydroxyl group of phosphatidylglycerol, carboxyl group of phosphatidylserine and the like; the amino group of phosphatidylethanolamine is preferably used. For the formation of a covalent bond between the reaction-active functional group of a phospholipid and PEG various chemistries known in the art may be employed, including reactions with cyanuric chloride, carbodiimide, acid anhydride, glutaraldehyde and the like.

In order to prepare a liposome with the PEG-bound phospholipid in the lipid layer, a PEG-bound phospholipid may uniformly be mixed with a liposome-forming lipid in advance, and the lipid mixture may be treated by a conventional method to form liposomes Mixing ratio of the PEG-bound phospholipid with the liposome-forming lipid is 0.1-50 mol %, preferably 0.5-20 mol % and more preferably 1-5 mol % in terms of the molar ratio to the phospholipid of the main component.

For pegylated or non-pegylated liposomes, the lipids may be first dissolved in an organic solvent, such as ethanol, t-butanol, mixtures thereof, etc., and gently heated (e.g., 60° C.-70° C.). To the dissolved lipids, a pre-heated aqueous solution may be added while vigorously mixing. For example, a solution containing 150-300 mM buffer may be added. Buffers that may be used include, but are not limited to, ammonium sulphate, citrate, maleate and glutamate. Following mixing, the resulting multilamellar vesicles (“MLVs”) may be heated and extruded through an extrusion device to convert the MLVs to unilamellar liposome vesicles. The organic solvent used initially to dissolve the lipids may be removed from the liposome preparation by dialysis, diafiltration, etc.

One or more therapeutic drugs and/or diagnostic agents may be entrapped in the liposomes using transmembrane pH gradient loading. By raising the pH of the solution external to the liposomes, a pH differential will exist across the liposome bilayer. Thus, a transmembrane potential is created across the liposome bilayer and the one or more therapeutic drug and/or diagnostic agent is loaded into the liposomes by means of the transmembrane potential.

Generally, the therapeutic drug and/or diagnostic agent to lipid ratio is about 0.01 to about 0.5 (wt/wt). In one embodiment, therapeutic drug and/or diagnostic agent to lipid ratio is about 0.1. In another embodiment, the therapeutic drug and/or diagnostic agent to lipid ratio is about 0.3. In one embodiment, vesicles are prepared with a transmembrane ion gradient, and incubated with a therapeutic drug and/or diagnostic agent that is a weak acid or base under conditions that result in encapsulation of the therapeutic agent or diagnostic agent. In another embodiment vesicles are prepared in the presence of the therapeutic drug and/or diagnostic agent and the unecapsulated material removed by dialysis, ion exchange chromatography, gel filtration chromatography, or diafiltration.

A preferred embodiment for loading is based upon U.S. Pat. No. 5,192,549 and involves removing ammonium from the external media. The result creates a transmembrane ammonium concentration gradient that induces a pH gradient. The drug is added to the vesicles, and “remote” loaded following incubation at elevated temperatures.

In a preferred embodiment, with an agent that is essentially impermeable (e.g., a diagnostic agent such as gadodiamide), the agent is present in the buffer that is used to make the liposomes and becomes passively encapsulated at the time of vesicle formation. This preferred method also applies to other zwitterionic drugs such as methotrexate. In contrast, weak bases (and acids) can be remote loaded into liposomes.

The liposomal formulations described herein may be used for CED to CNS regions, and CED can achieve high tissue distribution volumes within the CNS. Accordingly, the liposomal formulations may be used for the treatment of CNS disorders. Such CNS disorders include, but are not limited to CNS tumors such as, e.g., glioblastoma, astrocytoma, etc.

In a preferred embodiment, the first antineoplastic agent, e.g., topotecan, is administered as a liposomal formulation by CED. See, e.g., U.S. Patent Publication No. 20110274625.

### Oral Dosage Forms

Pharmaceutical compositions of the invention that are suitable for oral administration, e.g. TMZ, can be presented as discrete dosage forms, such as, but are not limited to, tablets (e.g., chewable tablets), caplets, capsules, and liquids (e.g., flavored syrups). Such dosage forms contain predetermined amounts of antineoplastic agents, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the TMZ in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the antineoplastic agents with liquid carriers, finely divided solid carriers, or both, and then shaping the product into the desired presentation if necessary.

Examples of excipients that can be used in oral dosage forms of the invention include, but are not limited to, binders, fillers, disintegrates, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Piano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Convection Enhanced Delivery

CED is a direct intracranial drug delivery technique that utilizes a bulk-flow mechanism to deliver and distribute macromolecules to clinically significant volumes of solid tissues. CED offers a greater volume of distribution than simple diffusion and is designed to direct a therapeutic drug to a specific target site. See, e.g., U.S. Pat. No. 5,720,720, the disclosure of which is expressly incorporated by reference herein. Briefly, CED is a method that circumvents the blood-brain barrier and allows large molecular weight substances, such as drug-loaded liposomes, to be administered uniformly and in a controlled fashion within a defined region of brain. (See for example, U.S. Ser. No. 11/740,548, incorporated herein in its entirety by reference). CED may be used to administer a fluid antineoplastic agent (e.g., in a liposomal formulation) to a solid tissue (e.g., a brain tumor) through direct convective interstitial infusion and over a predetermined time by inserting a catheter directly into the tissue; and administering the agent under pressure through the catheter into the interstitial space at a predetermined flow rate, e.g., from about 0.1 μL/min to about 12 μL/min.

A suitable apparatus that may be used for administration of a fluid antineoplastic agent (e.g., as pharmaceutical compositions) may comprise a pump device that contains a reservoir filled with the fluid antineoplastic agent. The pump may be external to the body or implanted within the body. The pump may be connected to a catheter, which may be implanted into discrete tissue(s) within the CNS. The pump may be activated to release the fluid antineoplastic agent at a pressure and flow rate that causes the solute to convect within the specific tissue.

The duration and other parameters of the infusion may be adjusted to distribute the fluid antineoplastic agent throughout the discrete tissue(s) to areas adjacent to the discrete tissue(s), although not into the cerebrospinal fluid. Depending upon the size and shape of the discrete tissue(s), it may be necessary to use multiple implanted infusion catheters or to use an infusion catheter with multiple solution exit ports.

Using CED, a fluid antineoplastic agent may be distributed by slow infusion into the interstitial space under positive pressure through a fine cannula. Bulk flow driven by hydrostatic pressure derived from a pump may be used to distribute the fluid antineoplastic agent within the extracellular spaces of the CNS. Because the use of CED permits distribution of fluid antineoplastic agents directly within nervous tissues via the tip of a cannula, the blood-brain barrier is bypassed and discrete tissues in the CNS may be targeted, including discrete tissue defined, e.g., as cancerous or identified as for resection by a conventional presurgical evaluation, and in different foci if more than one focus are in need of treatment. Based on the properties of bulk flow, CED may be used to distribute fluid antineoplastic agents reliably, safely, and homogeneously over a range of volumes. See for example U.S. Ser. No. 11/740,508. Further, CED does not cause structural or functional damage to the infused tissue and provides greater control over the distribution of the fluid antineoplastic agent. Additionally, fluid antineoplastic agents in a liposomal formulation may be distributed homogeneously throughout a distribution volume that is proportional to the infusion volume regardless of the molecular weight of the liposomes comprised in the liposomal formulations.

In one embodiment, a delivery system comprising an ultrafine delivery catheter (constructed of polyurethane and fused silica in a novel “step” design, for example as described below) that in some embodiments is subcutaneously connected with a transcutaneous port may be implanted. The delivery system may be rapidly biointegrable and may be internally sealed and filtered to prevent bacterial ingress and capped for further safety. A fluid antineoplastic agent may be infused as needed through the port of this catheter system.

In one embodiment described herein, CED may be applied using an infusion pump with a small diameter catheter permanently implanted in the brain region. Fluid antineoplastic agents to be administered may be prepared as an aqueous isotonic solution, or other appropriate formulation. During the administration (e.g., infusion), the liposomal solution may flow within the extracellular space and cause minimal to no damage to the brain tissue.

In one embodiment, an ultrafine (0.2 mm OD at tip), minimally traumatic catheter system specially designed for transcutaneous CED delivery is used. The catheter system has a step design, which may eliminate solution reflux along the sides of the catheter. Such solution leakage is a major problem with straight-sided catheters. The catheter system may be constructed of polyurethane and fused silica or Peek Optima so that it is highly biocompatible and does not interfere with MRI signals. Treatment of CNS disorders may require readministration of a fluid antineoplastic agent at varying intervals, e.g., weekly intervals, monthly intervals, etc. For example, see U.S. Ser. No. 11/740,124, the disclosure of which is expressly incorporated by reference herein. The optional transcutaneous port, if present, may remain capped during the interval period. Using multiple catheters is feasible so that it may be possible to perfuse a larger area of discrete tissue(s) than is feasible with a single catheter. It has been found that the volume of distribution of liposomes after CED infusion is linearly related to the solution volume infused.

An especially preferred cannula is disclosed in Krauze et al., J Neurosurg. November 2005; 103(5):923-9, incorporated herein by reference in its entirety, as well as in U.S. Patent Application Publication No. US 2007/0088295 A1, incorporated herein by reference in its entirety, and United States Patent Application Publication No. US 2006/0135945 A1, incorporated herein by reference in its entirety. In one embodiment, CED comprises an infusion rate of between about 0.1 μL/min and about 10 μL/min. In another embodiment, CED comprises an infusion rate of greater than about 0.5 μL/min, more preferably greater than about 0.7 μL/min, more preferably greater than about 1 μL/min, more preferably greater than about 1.2 μL/min, more preferably greater than about 1.5 μL/min, more preferably greater than about 1.7 μL/min, more preferably greater than about 2 μL/min, more preferably greater than about 2.2 μL/min, more preferably greater than about 2.5 μL/min, more preferably greater than about 2.7 μL/min, more preferably greater than about 3 μL/min, and preferably less than about 12 μL/min, more preferably less than about 10 μL/min.

In a preferred embodiment, CED comprises incremental increases in flow rate, referred to as “stepping” or up-titration, during delivery. Preferably, stepping comprises infusion rates of between about 0.1 μL/min and about 10 μL/min.

In a preferred embodiment, stepping comprises infusion rates of greater than about 0.5 μL/min, more preferably greater than about 0.7 μL/min, more preferably greater than about 1 μL/min, more preferably greater than about 1.2 μL/min, more preferably greater than about 1.5 μL/min, more preferably greater than about 1.7 μL/min, more preferably greater than about 2 μL/min, more preferably greater than about 2.2 μL/min, more preferably greater than about 2.5 μL/min, more preferably greater than about 2.7 μL/min, more preferably greater than about 3 μL/min, and preferably less than about 12 μL/min, more preferably less than about 10 μL/min.

In a preferred embodiment, CED comprises continuous increases in flow rate, referred to as “ramping” or up-titration, during delivery. Preferably, ramping comprises infusion rates of between about 0.0 μL/min and about 10 μL/min.

In a preferred embodiment, ramping comprises infusion rates of greater than about 0.5 μL/min, more preferably greater than about 0.7 μL/min, more preferably greater than about 1 μL/min, more preferably greater than about 1.2 μL/min, more preferably greater than about 1.5 μL/min, more preferably greater than about 1.7 μL/min, more preferably greater than about 2 μL/min, more preferably greater than about 2.2 μL/min, more preferably greater than about 2.5 μL/min, more preferably greater than about 2.7 μL/min, more preferably greater than about 3 μL/min, and preferably less than about 12 μL/min, more preferably less than about 10 μL/min.

### Concomitant Delivery

The term “concomitant delivery,” “delivered concomitantly,” or “concomitant therapy” is used when the at least two antineoplastic agents are given concurrently, i.e., either at the same time or within the same period of time as each other regardless of the delivery methods. Such concomitant delivery allows the first and second antineoplastic agents to provide a synergistic therapeutic effect that is not seen when each of the antineoplastic agents are delivered alone. Concomitant administration is performed for a period of time, e.g. a single administration dose, multiple administration doses, a defined regimen of scheduled doses, etc. An appropriate period of time may be 1 day, 2 days, 3 days, 4 days, 5 days, 6 days, 1 week, 2 weeks, 3 weeks, 4 weeks, 5 weeks, 6 weeks, 1 month, 2 months, 3 months, 4 months, 5 months, 6 months, 7 months, 8 months, etc.

In specific embodiments of the invention, TMZ may be administered systemically or by CED over an extended period of time, e.g. in accordance with current protocols. During at least a portion of the time in which TMZ is administered, the liposomally encapsulated topoisomerase I inhibitor administered by CED is concomitantly administered, including without limitation TopoCED™. The concomitant administration may be during all or part of the initial phase of treatment, e.g. the initial week, 2 week, 3 week, 4 week, 5 week, 6 week, etc. phase; during all or part of the maintenance phase, e.g. following the initial phase and an optional pause in treatment and during any or all of the cycles of maintenance treatment; or both the initial phase and the maintenance phase. Additional therapeutic regimens are not excluded, e.g. a concomitant initiation phase may also comprise radiation, other chemotherapeutic agents; and the like.

All patents and patent publications referred to herein are hereby incorporated by reference.

Certain modifications and improvements will occur to those skilled in the art upon a reading of the foregoing description. It should be understood that all such modifications and improvements have been deleted herein for the sake of conciseness and readability but are properly within the scope of the following claims.

### EXPERIMENTAL

### Example 1

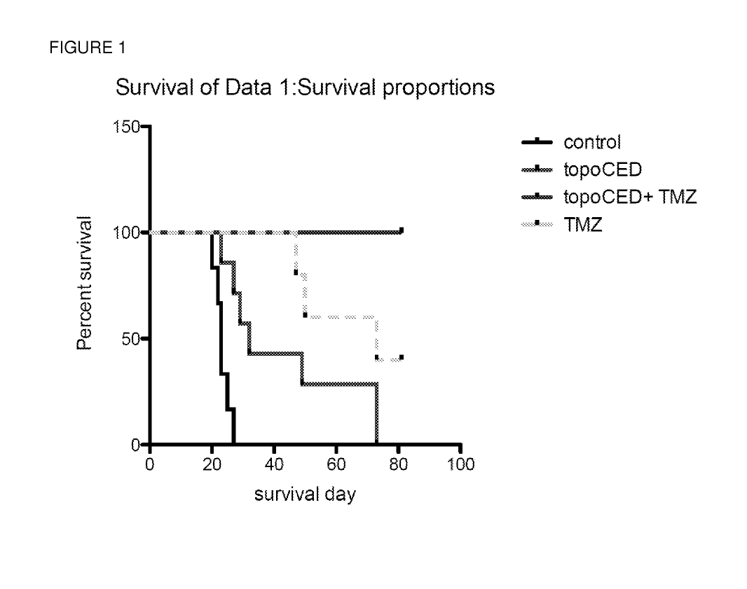

A synergistic combined therapy for treatment of glioblastoma was obtained by CED of a convectable non-PEGylated liposomal formulation encapsulating the topoisomerase I inhibitor topotecan (topoCED™); with systemic delivery of TMZ. In animal studies, the combined therapy provided for an increased lifespan of animals in a xenograft model for a human tumor, as shown in FIG. 1. Significantly, the tumors literally melted away in 5 of the 6 animals treated by the subject combination therapy, and only residual tumor was found in the 6^{th} animal.

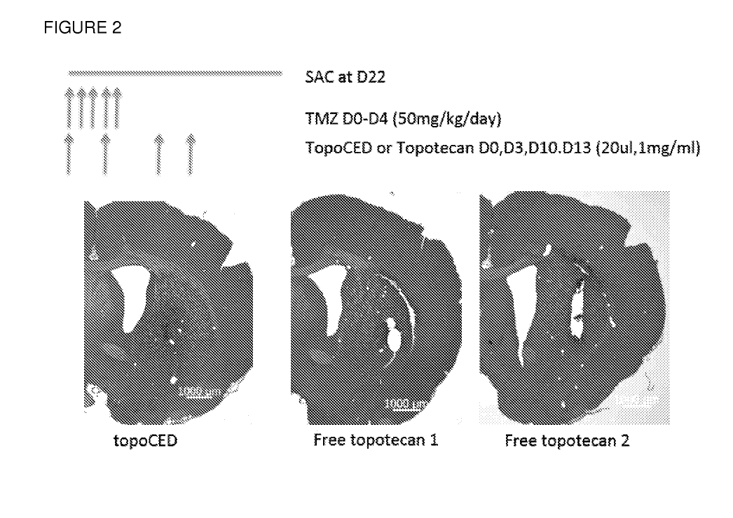

Topotecan has been previously tested in a number of clinical studies as a systemic agent combined with radiotherapy; or paclitaxel. Overall, the results of these studies suggest that delivering a large enough concentration of systemic topotecan to kill the tumor cells results in unacceptable systemic toxicity. By infusing the tumor with TopoCED™, instead of the free drug, the toxicity is greatly reduced, as shown in FIG. 2.

In conclusion, a combination of liposomally encapsulated topotecan and systemic TMZ provides significant efficacy against glioblastoma using the in vivo U87MG intracranial rodent xenograft model.

### Materials and Methods

Liposomes were composed of distearoylphosphatidylcholine (DSPC), distearoylphosphatidylglycerol (DSPG) and cholesterol (chol), prepared by dissolution of all lipids in t-butanol/ethanol/water, heated, then added to a solution of ammonium sulphate to generate multilamellar vesicles (MLVs). The MLVs were extruded to yield large unilamellar vesicles (LUVs), then concentrated by ultrafiltration and subsequently diafiltered to remove the solvent and exchange the buffer. Topotecan was loaded into the liposomes by addition of a solution to a suspension of LUVs, resulting in a topotecan concentration of about 1 mg/ml.

For determining tissue concentrations of TMZ, adult male Sprague-Dawley rats were treated as stated. The animals were sacrificed at the indicated time points. The brains were removed, placed on ice, the brain dissected and the tissue homogenized and frozen, then analyzed for drug concentration using a validated reversed phase HPLC method.

Human glioblastoma multiforme cell line U87MG was used for xenograft implant experiments. Cells were maintained as monolayers in Eagle's minimal essential medium supplemented with 10% fetal calf serum, antibiotics (streptomycin 100 μg/ml, penicillin 100 U/ml), and nonessential amino acids. Cells were cultured at 37° C. in a humidified atmosphere of 95% air and 5% carbon dioxide. Cells were to be harvested on the day of tumor inoculation surgery.

Congenitally athymic, male, homozygotic, nude rats were housed under aseptic conditions. For the intracranial xenograft tumor model, U87MG cells as described earlier were harvested on the day of tumor inoculation and resuspended in Hank's balanced salt solution without Ca²⁺ and Mg²⁺ (HBSS) for implantation. A target cell suspension was implanted unilaterally into the right striatal region of the athymic rat brains. Under isoflurane anesthesia, rats were mounted in a stereotaxic frame (David Kopf Instruments, Tujunga, Calif., USA) with the head positioned by ear bars and the incisor bar. A longitudinal incision was made in the skin on top of the skull and blunt dissection was used to remove connective tissue overlying the skull. A burr-hole was drilled 0.5 mm anterior and 3.0 mm lateral from the bregma. U87MG cell suspension was stereotaxically injected into the right striatum using the appropriate dorso-ventral coordinates from pial surface. Following inoculation, the skin was stapled. The survival time following implantation in the absence of treatment was expected to be approximately 0-30 days.

### Example 2

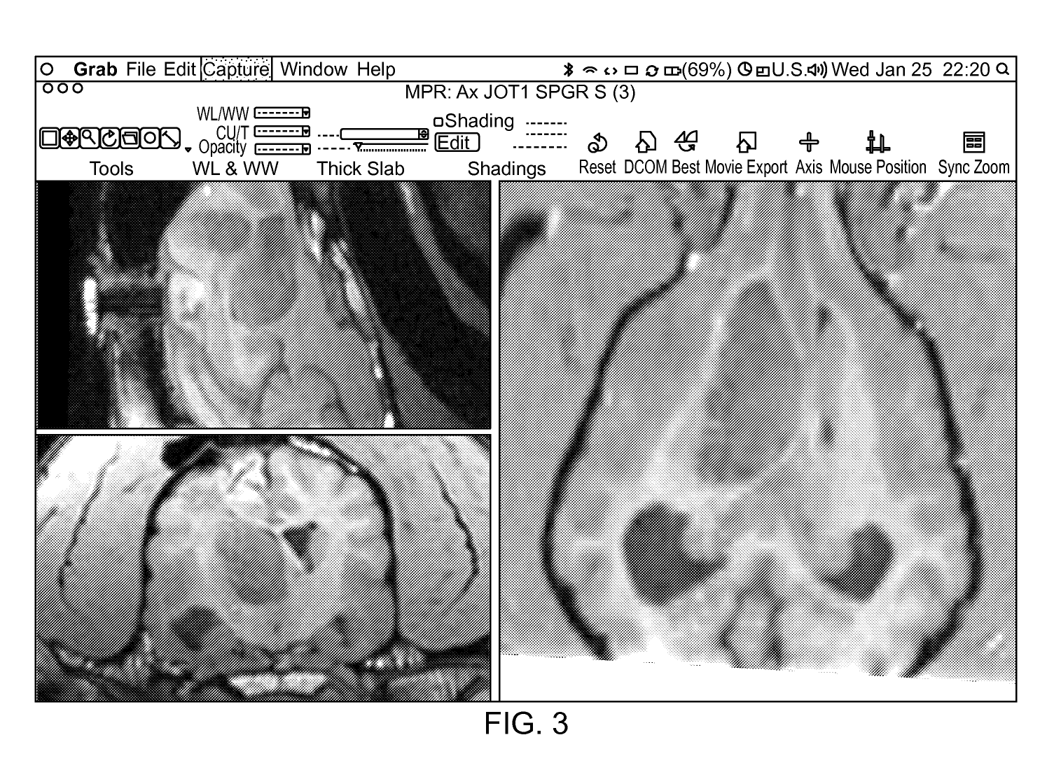

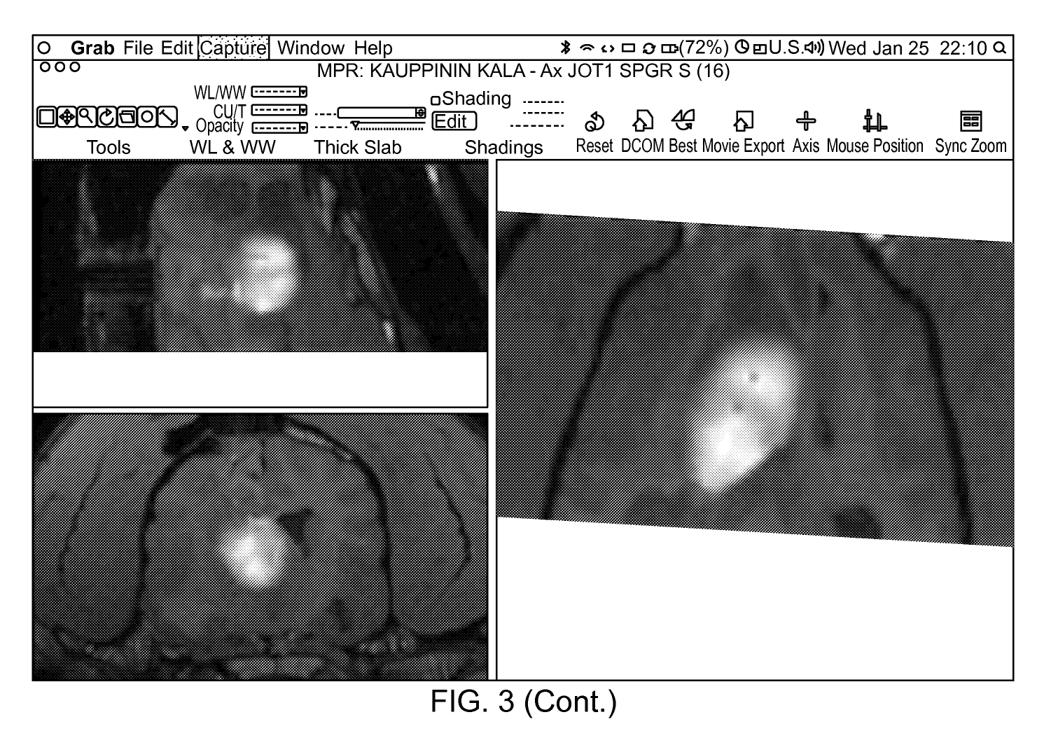

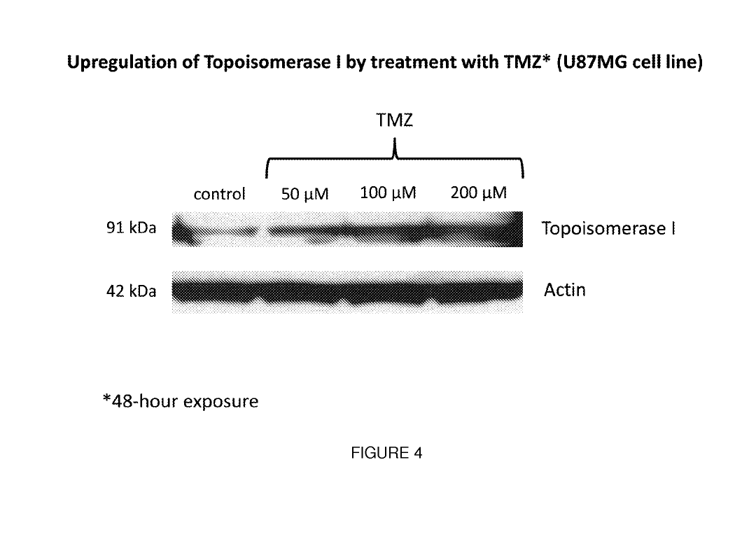

CED of TopoCED™ in a canine astrocytoma grade III. Shown in FIG. 3, the largely infused hyperintense area (grey circle) in the T2-weighted image containing the tumor epicenter was located in the caudate nucleus (A). Two areas (encircled in black) containing tumor cells were only minimally infused. The corresponding LFB and HE stained brain sections were examined by light microscopy (B) in order to compare the presence of neoplastic cells in infused versus non-infused areas. Neoplastic cells diminished dramatically in infused areas (C). Neoplastic cells in poorly infused areas were high in numbers and organized as a solid proliferating tumor (D). These marked differences in cell proliferation were highlighted by the reactivity of cells to MIB-1 antibodies.

### Example 3

Human glioblastoma multiforme cell line U87MG was maintained as monolayers in Eagle's minimal essential medium supplemented with 10% fetal calf serum, antibiotics (streptomycin 100 ug/ml, penicillin 100 U/ml), and nonessential amino acids. Cells were cultured at 37° C. in a humidified atmosphere of 95% air and 5% carbon dioxide.

Cells were exposed to TMZ at a concentration of from 50-200 μM for a period of 48 hours, then lysed, immunoprecipitated and run on a gel. The results are shown in FIG. 7 and demonstrate a clear upregulation in topoisomerase I expression with increasing TMZ concentrations. This upregulation provides a compelling explanation for the synergy between TMZ and topoisomerase I inhibitors such as topotecan, although it is important to note that no such synergy has ever been observed in vivo before the concomitant use of systemically administered TMZ with TopoCED™ administered via CED as described in Example 1.

1. A method of treating a central nervous system (CNS) tumor in a patient in need thereof comprising:
administering to the patient by convection enhanced delivery (CED) a therapeutically effective amount of a topoisomerase inhibitor encapsulated in a liposome; and
a therapeutically effective dose of an alkylating agent.

2. The method of claim 1, wherein the CNS tumor is a glioma.

3. The method of claim 2, wherein the glioma is glioblastoma multiforme.

4. The method of claim 2, wherein the glioma is anaplastic astrocytoma.

5. The method of claim 2; wherein the glioma is oligodendroglioma.

6. The method of claim 1, wherein the topoisomerase inhibitor is camptothecan or a derivative thereof.

7. The method of claim 6, wherein the topoisomerase inhibitor is topotecan.

8. The method of claim 1, wherein the alkylating agent is temozolomide or dacarbazine.

9. The method of claim 8, wherein the alkylating agent is temozolomide.

10. The method of claim 9, wherein the temozolomide is administered orally.

11. The method of claim 9, wherein the temozolomide is administered by CED.

12. The method of any of the preceding claims, wherein the topoisomerase inhibitor and alkylating agent are delivered concomitantly for a period of time.

13. The method of any of the preceding claims, wherein the topoisomerase inhibitor is concomitantly administered during at least a portion of the period of time in which the alkylating agent is administered.

14. The method of claim 12 or claim 13, wherein the period of time for concomitant administration is all or part of initial phase of treatment.

15. The method of claim 12 or claim 13, wherein the period of time for concomitant administration is all or part of maintenance phase of treatment.

16. The method of claim 12 or claim 13, wherein the period of time for concomitant administration is all or part of both initial and maintenance phase of treatment.

17. The method of any of the preceding claims, wherein the combination provides for a synergistic effect.
